(19) United States
(12) Patent Application Publication
(10) Pub. No.: US 2005/0209616 A1
(43) Pub. Date: Sep. 22, 2005

(54) Catheter and method for use of such a catheter for removing a stenosis from a vessel

(75) Inventor: Nicolaas Johannes Dongelmans, Hoornaar (NL)

Correspondence Address: ALTERA LAW GROUP, LLC, 6500 CITY WEST PARKWAY, SUITE 100, MINNEAPOLIS, MN, 55344-7704, US

(73) Assignee: Smart Medical Solutions B.V., Gorinchem (NL)

(21) Appl. No.: 10/867,274

(22) Filed: Jun. 14, 2004

(30) Foreign Application Priority Data
Mar. 22, 2004 (NL) 1025780

(51) Int. Cl. A61B 17/22

(52) U.S. Cl. 606/159

(57) ABSTRACT

A method and catheter for removing a stenosis from a vessel, wherein the catheter is provided with a shaft (1) having a proximal (1′) and distal (1″) end, and with a stenosis-removing part (2) near the distal end (1″) of the shaft (1), wherein the stenosis-removing part (2) is provided with hairs (3) which can be brought into a first position and a second position, wherein, in a first position, the hairs (3) substantially extend inside the circumferential contours of the shaft (1), wherein, in a second position, the hairs (3) extend outside the shaft (1), so that the radial ends of the hairs (3) are at a greater radial distance from the center line of the shaft (1) than in the first position, wherein means (5, 6, 12; 6, 12 14, 15, 16, 17) are provided for making the hairs (3) vibrate at a high frequency.

The invention relates to a catheter for removing a stenosis from a blood vessel. The invention also relates to a method for removing a stenosis from a blood vessel by using such a catheter.

Since the early eighties, it is sought to dilate vasoconstrictions in patients with stenoses by means of balloon catheters. The drawback of this treatment is that, in 15 to 60 percent of the treated patients, after a few months, a new constriction arises at the location of the treatment (restenosis). In order to solve this problem, so-called stents have been developed which should prevent the formation of a new stenosis. It was found in practice that this did not provide sufficient solace either. Then, so-called drug-eluting stents were developed, which are provided with a coating which ensures the prolonged release of a restenosis-limiting substance. A disadvantage of this new application is the relatively high cost price of the stents, of which usually 2 to 3 are placed per patient. In addition, little is as yet known about the consequences for the body of the medicines present in the coating, which are released gradually and prolongedly. There are doubts about the damaging effects thereof, especially in combination with the complex of remedies already administered to patients with the respective symptoms. Despite the fact that encouraging results are achieved with the drug-eluting stents, considerable drawbacks of the use of the balloon catheter still remain.

Firstly, a great drawback is that, for dilating the obstructed blood vessel, the inflated balloon completely closes off the blood vessel during the treatment, with the risk of a further damage of the downstream tissue, such as for instance heart tissue.

A second drawback is the damage of the blood vessel as a result of pushing the stenosis outwards. This creates, as it were, a bump in the blood vessel with damages and tears in the vascular wall. Therefore, no catheter treatment with a balloon catheter is without risks. A certain percentage with a fatal outcome is inevitable with the existing technique, possibly as a result of an infarct by the complete closure of the blood supply during the operation or as a result of other acutely arising problems.

A third drawback of balloon catheterization with placement of stents is that this treatment is not possible in the case of diffuse and longer constrictions, for branches and tortuous vessels, and for vessels with a small diameter in general.

The present invention contemplates a catheter and a method for removing a stenosis in a blood vessel, by which the safety of the patient is better guaranteed, both by preventing problems during the treatment itself and by dealing with problems which cannot be solved with a balloon catheter, optionally in combination with a stent.

For this purpose, the invention provides a catheter provided with a shaft having a proximal and distal end, and provided with a stenosis-removing part located near the distal end of the shaft, with the stenosis-removing part being provided with hairs which can be brought into a first position and a second position, while, in a first position, the hairs extend substantially inside the circumferential contours of the shaft, while, in a second position, the hairs extend outside the circumferential contours of the shaft, so that the radial ends of the hairs are at a greater radial distance from the center line of the shaft than in the first position, while means are provided for making the hairs vibrate at a high frequency.

Further, the invention provides a method for treating a stenosis in a blood vessel, with a catheter according to the invention with an insertion sleeve being slid into the blood vessel until the stenosis-removing part is near the stenosis, the insertion sleeve then being removed from the stenosis-removing part, so that the hairs proceed from the first position to the second position, with, then, the means for making the hairs vibrate at a high frequency being switched on.

Compared to the balloon catheter used up to now, the following advantages are achieved:
there is no closure of the blood vessel during the treatment and hence no risk of an infarct;
the stenosis is removed instead of pushed away;
the blood vessel is not stretched, so no vascular wall damages or tears are created;
the placement of stents becomes unnecessary, which results in considerable cost savings;
the catheter is also suitable for cleaning previously placed stents;
the patients are not excessively burdened by administration of restenosis-preventing medicines, like with the drug-eluting stent.

In addition, the catheter according to the invention is particularly suitable for various vascular diameters from 0.1 mm. Further, the catheter according to the invention allows treatment of various types of stenosis, including short, long and diffuse stenoses. Because the stenosis is actually removed, the percentage of restenosis occurring is expected to be considerably lower compared to balloon catheterization.

According to a further elaboration of the invention, the means for making the hairs vibrate at a high frequency are designed for subjecting the hairs to a vibration frequency which is in the range of 10-100,000 Hz, more in particular in the range of 1000-50,000 Hz.

In addition, it is possible to vary the frequency during the treatment to achieve an optimal result. With the frequencies mentioned, a stenosis can be removed in an effective manner.

The catheter is used as follows. First, in a manner known per se, the vessel is accessed, which is a standard procedure in, for instance, PTA (percutaneous transluminal angioplasty) and PTCA (percutaneous transluminal coronary angioplasty) procedures. Then, a catheter is slid into the vessel until the stenosis-removing part is near the distal end of the stenosis. When the catheter is of the “over-the-wire” type, first, a so-called guide wire is slid into the vessel until a distal end thereof has been slid beyond the stenosis. Over this guide wire, then, the catheter is slid, which catheter is provided with a first lumen for this purpose. After this, the hairs are brought from the first position, in which they extend substantially inside the circumferential contours of the shaft, to the second position, so that the radial ends of the hairs are at a greater distance from the center line of the shaft than in the first position. Then, the means for making the hairs vibrate at a high frequency are switched on. As a result of this, the hairs will vibrate the stenosis material loose from the vascular wall. In practice, it has been found that, when the hairs are made to vibrate at a high frequency, they remove the stenosis material from the vascular wall in a highly effective manner, while the vascular wall is not or hardly damaged during this. During the vibrating, the catheter can slowly be withdrawn in proximal direction, until the whole stenosis has been removed.

Preferably, proximal and optionally also distal of the stenosis-removing part, a filter has been provided. With such a filter, the stenosis material vibrated loose can be blocked, so that this material is prevented from causing another obstruction further downstream. Optionally, according to a further elaboration of the invention, distal of the proximal filter, a second and a third lumen may also terminate for discharging stenosis material vibrated loose and for supplying washing fluid, respectively.

According to a further elaboration of the invention, the catheter may be provided with measuring means for measuring the vibration of the hairs and/or holder, while evaluation means are provided for evaluation the measurements, the evaluation means being connected to the frequency generator and the evaluation results determining the frequency and/or amplitude to be generated by the frequency generator. With such a feedback of the vibration realized during the treatment, an optimal cleaning result can be obtained within a minimal period of time.

According to a further elaboration of the method according to the invention, during the treatment, the result achieved during the treatment may also be determined, for instance by means of X-ray images or ultrasound, with, depending on this determination, the treatment being continued, or terminated, or changed by changing frequency and/or amplitude of the vibration of by replacing the catheter with a catheter having a different diameter.

After the whole stenosis has been treated, the catheter with the guide wire, if any, can be removed from the blood vessel. The invention will now be further elucidated on the basis of two exemplary embodiments, with reference to the drawing, in which:

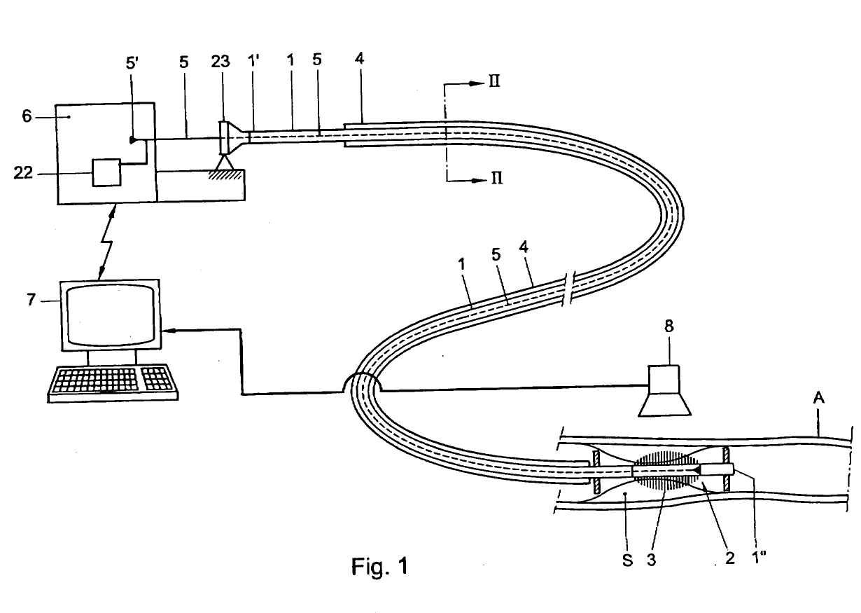

FIG. 1 shows a diagrammatic side elevational view of a first exemplary embodiment of the catheter;

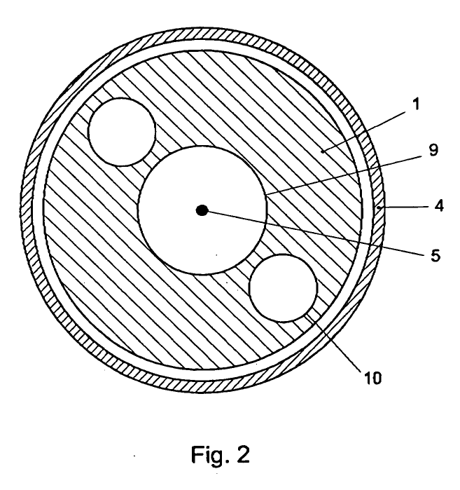

FIG. 2 shows a cross-sectional view over line II-II of FIG. 1;

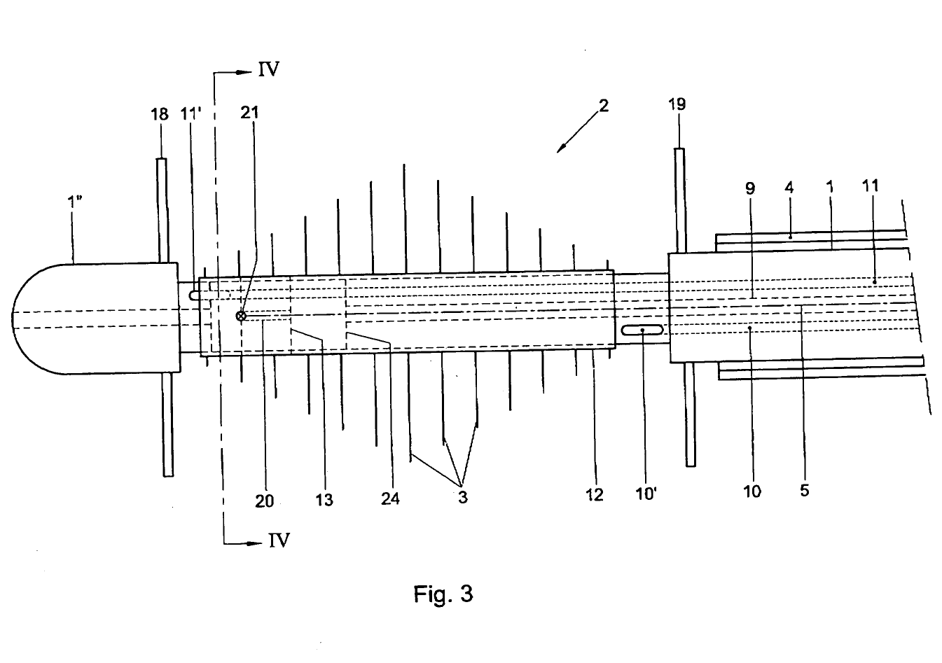

FIG. 3 shows, in more detail, the stenosis-removing part of the catheter shown in FIG. 1;

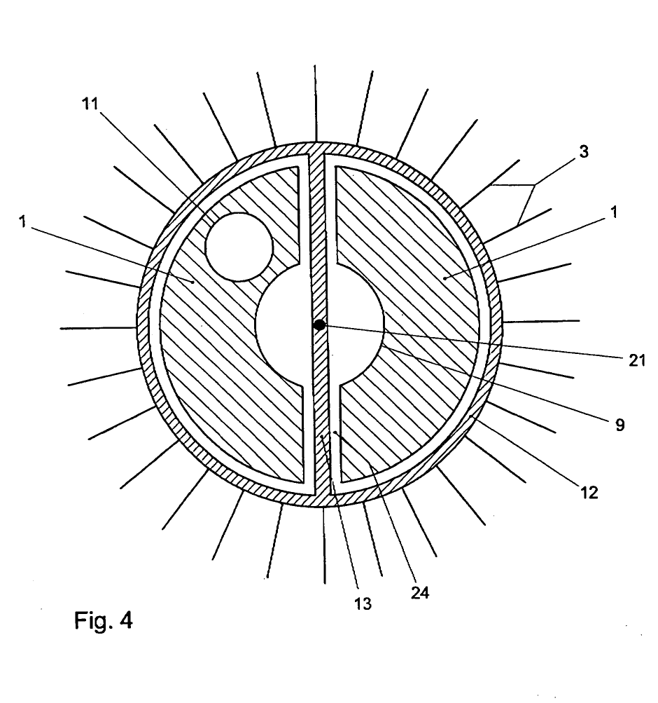

FIG. 4 shows a cross-sectional view over line IV-IV of FIG. 3;

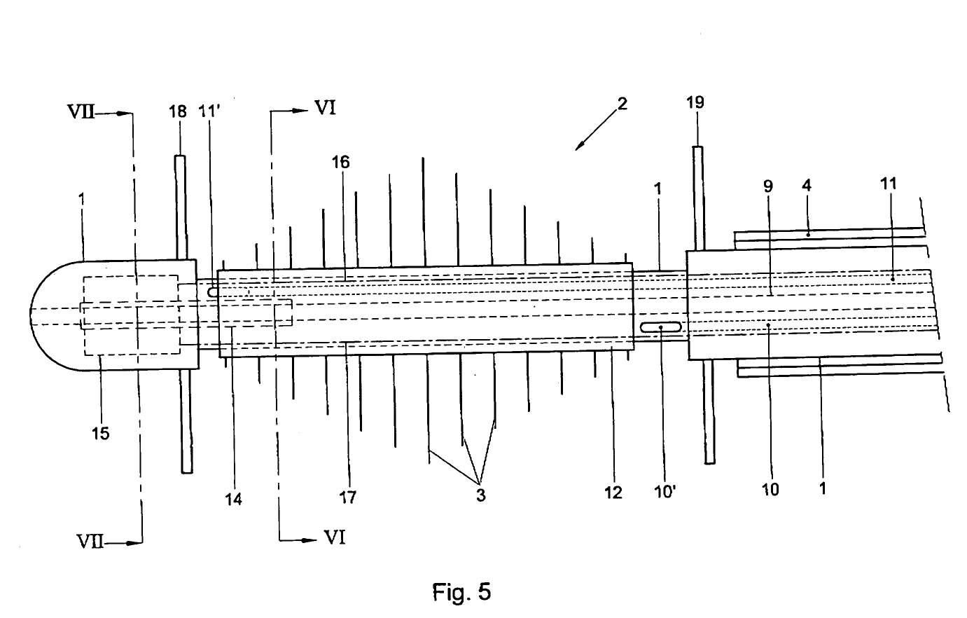

FIG. 5 shows the stenosis-removing part of a second exemplary embodiment;

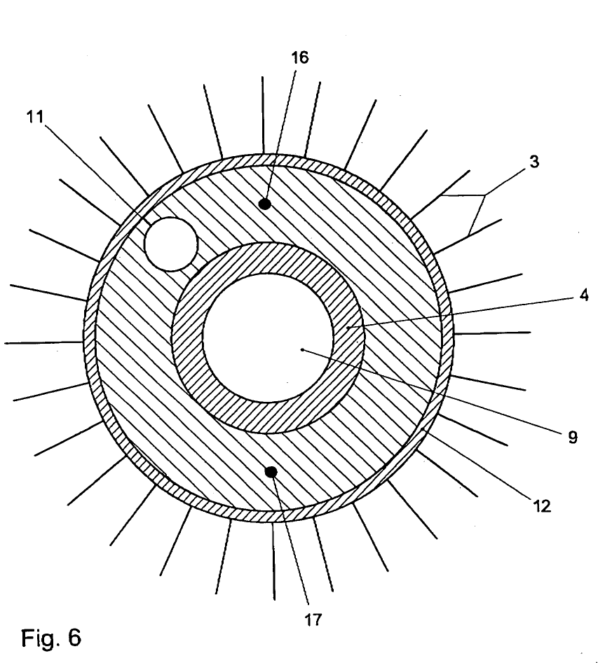

FIG. 6 shows a cross-sectional view over line VI-VI of FIG. 5; and

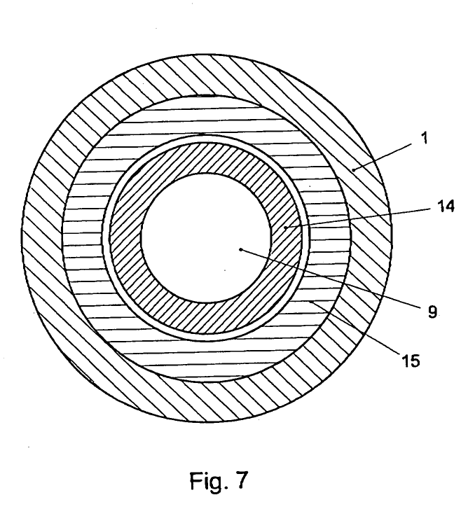

FIG. 7 shows a cross-sectional view over line VII-VII of FIG. 5.

It is noted that the Figures are by no means to scale.

FIG. 1 clearly shows a catheter of which the main part is formed by the catheter shaft 1. Near the distal end 1″ of the shaft 1, the catheter is provided with a stenosis removing part 2. The stenosis-removing part 2, which will hereinafter be discussed in more detail with reference to FIGS. 3 and 5, is inter alia provided with hairs 3 which can be brought into high-frequent vibration. Over the shaft 1, an insertion sleeve 4 has been provided. By means of the insertion sleeve 4, which is slidable over the shaft 1, the hairs 3 can be brought into a first position, in which these hairs 3 extend substantially inside the circumferential contours of the shaft 1. By sliding the insertion sleeve 4 over the shaft 1 in proximal direction, the hairs 3 are released and will proceed into a second position. In this second position, the hairs 3 extend outside the circumferential contours is of the shaft 1, so that the radial ends of the hairs are at a greater radial distance from the center line of the shaft 1 than in the first position. In the exemplary embodiment of FIG. 1, the shaft 1 is provided with a first lumen 9 through which a guide wire 5 extends. The lumen 9 is clearly visible in a cross-sectional view of FIG. 2. The shaft 1 is movable in axial direction over the guide wire 5.

In the exemplary embodiment of FIG. 1, the proximal end 1′ of the shaft 1 is connected with a frequency generator 6. In the Figure, this is diagrammatically shown by diagrammatically connecting the Luer coupling 23 with the frequency generator 6. A proximal end 5′ of the guide wire 5 is connected with a frequency-generating element (not shown) in the frequency generator 6.

In addition to the elements discussed hereinabove, FIG. 2 also shows a second lumen 10 and a third lumen 11. The functions of these lumens 10, 11 will be returned to later.

The distal end of the catheter shown in FIG. 3 shows the shaft 1 through which the first lumen 9, the second lumen 10 and the third lumen 11 extend. Also, the guide wire 5 is shown as a dotted line, which guide wire 5 is provided with a coupling element 21 at its distal end. The coupling element 21 can engage a cross wall or cross pin 13 which is part of a bush-shaped holder 12 carrying the brush hairs 3. The holder 12 is connected to the shaft 1 so as to be slidable in axial direction. The cross wall or pin 13 extends through an axial slot 24 in the shaft 1. This axial slot 24 allows the axial movement of the holder 12 with respect to the shaft 1. The cross wall or pin 13 forms the point of engagement for the coupling element 21 which is connected with the distal end of the guide wire 5. When, by means of the frequency generator 6, the guide wire 5 is subjected to a high-frequency vibration, this vibration is transmitted to the holder 12 and, accordingly, the hairs 3 via the coupling element 21 and the cross wall or pin 13. Distal of the holder 12, a filter 18 has been provided. Proximal of the holder 12, a filter 19 has been provided. These filters are also kept in a folded position by the insertion sleeve 4. The space bounded by the distal filter 18 and the proximal filter can be exhausted during the treatment via the second lumen 10 of which a distal opening 10′ is shown in FIG. 3. Optionally, via the third lumen 11, washing fluid can be supplied to the space. The third lumen terminates in the respective space via opening 11′ which is also shown in FIG. 3. It will be clear that the filters 18, 19 preferably have a good fluid permeability, so that the blood can easily pass the respective filters 18, 19. On the other hand, the filters do need to block the coarser released stenosis material. It is noted that they filters 18, 19 are not strictly necessary. It is also possible to provide only one filter arranged on the downstream side of the stenosis-removing part. Usually, this will be the proximal filter 19 and the distal filter 18 can be omitted.

FIG. 4 again clearly shows the manner in which the holder 12 with cross wall or pin 13 cooperates with the shaft 1 and the axial slot 24 present therein. Further, the third lumen 11 in the shaft 1 is clearly shown in FIG. 4.

FIGS. 5-7 show a second exemplary embodiment of the distal end of a catheter according to the invention. In these Figures, the shaft 1 is also clearly visible, with first lumen 9, second lumen 10 and third lumen 11 extending therein. Again, the outlet opening 10′ of the second lumen 10 and the outlet opening 11′ of the third lumen 11 are clearly shown. Near the stenosis-removing part 2, the shaft 1 is provided with a reduced diameter. At the location of this reduced diameter, a bush-shaped holder 12 has been mounted on the shaft 1 so as to be slidable in axial direction. The holder 12 carries hairs 3 which can be brought into high-frequency vibration by axial movement of the holder 12 over the shaft. In the present exemplary embodiment, this axial movement is realized by an actuator designed as a coil 15. The actuator 15 has been connected to a frequency generator 6 via two electrical conductors 16, 17 extending through the shaft 1. In the coil 15, a core 14 extends. Preferably, the holder 12 is at least partly manufactured from ferromagnetic material, such that an alternating magnetic field exerted by the core 14 as a result of an alternating magnetic field in the coil 15 results in an axial movement of the holder 12. By varying the magnetic field at a high frequency, thus, the hairs 3 can be subjected to a high-frequency vibration. Optionally, the electrical conductors 16, 17 may also serve to measure the vibration of the hairs 3 and/or holder 12. Such measurement data can be sent to evaluation means 7. The evaluation means 7 can evaluate the respective measurements and, depending on the evaluation results, determine the frequency and/or amplitude generated by the frequency generator 6. In the exemplary embodiment shown, the coil 15 is part of the shaft of the catheter. However, it is also possible for the coil to be accommodated in a separate sleeve which is slid over the catheter shaft when the hairs 3 need to be brought into vibration, such that the coil is near the holder 12 on the catheter shaft.

In the exemplary embodiment of FIG. 1, the measuring means are inter alia formed by a sensor 22 which is included in the frequency generator 6 and which measures the vibration in the guide wire 5. The measurements observed by the sensor 22 can be fed to evaluation means 7 for determining a desired frequency and/or amplitude. Further, as shown in FIG. 1, further measuring means 8 may be provided, such as for instance means for observing X-ray images or for carrying out ultrasound. With such measuring means 8, the result achieved during the treatment can be determined. Depending on this determination, the treatment can be continued, or terminated, or changed by changing the frequency and/or amplitude of the vibration or by replacing the catheter with a catheter having a different diameter. For this purpose, the measuring means 8 may optionally be in communicative connection with the evaluation means 7. Of course, the physician may also take various actions on the basis of the images observed by the measuring means 8. Optionally, the physician may be supported by the evaluation means 7 in decision-making.

The operation of the device will now be further explained with reference to FIG. 1. FIG. 1 shows a part of a vessel A with a stenosis S therein. With an “over-the-wire” catheter, first, guide wire 5 will be fed into the vessel A beyond the stenosis S. Then, the shaft 1 of the catheter with the insertion sleeve 4 provided over it will be slid over the guide wire 5. Here, the distal end of the insertion sleeve is beyond the stenosis-removing part 2, so that the hairs 3 are in the first position and thus extend substantially inside the circumferential contours of the shaft 1. When the stenosis-removing part 2 is near the distal part of the stenosis S, the insertion sleeve 4 can be slid in proximal direction with respect to the shaft 1, such that the hairs 3 assume the second position, and the filters 18, 19 assume the unfolded position. Then, via the guide wire 5 or via the electrical conductors 16, 17, the coil 15 and core 14, and the holder 12 are brought into a high-frequency vibration, such that the hairs 3 are brought into a high-frequency vibration. The radial ends of the hairs 3 will pulverize the stenosis S and the released stenosis material can be exhausted via the second lumen 10. Optionally, washing fluid may be supplied via the third lumen 11 in order to promote the discharge of the released stenosis material. Depending on vibration measurements done via the electrical conductors 16, 17 or via the sensor 22 and processed by the evaluation means 7, the frequency and/or amplitude of the vibration may be varied. Further, optionally, depending on the result observed, which result may, for instance, be observed by means of X-ray images or ultrasound, it may be decided to continue the treatment, to terminate it or to change it by changing the frequency and/or amplitude of the vibration or by replacing the catheter with a catheter having a different diameter. During the treatment, the shaft 1 will slowly be moved in proximal direction with respect to the vessel A, so that, gradually, the whole stenosis S from the distal end to the proximal end thereof has been removed. After the removal of the stenosis S, the catheter shaft 1 can be removed from the vessel A.

The whole procedure is carried out without closure of the vessel A, so that the downstream tissue parts are prevented from temporarily not getting any blood supply. Because the stenosis material is actually removed instead of pushed way, like in balloon catheterization, tears and similar vascular wall damages are minimized.

Although, in the above, the procedure has been described with reference to an “over-the-wire” catheter, it will be clear to a skilled person that, also with a so-called wireless catheter, the concept of the invention can advantageously be used. Here, the catheter shaft 1 itself will serve to find the path to the stenosis. Optionally, for this purpose, the shaft may be provided, at its distal end, with a fixed piece of guide wire which is fixedly connected with the distal end 1″ of the shaft 1. In such an embodiment, the vibration transmission means are preferably designed in the manner as shown in FIGS. 5-7. This is because, in a “wireless” catheter, there is no guide wire which can be coupled with the holder 12 via coupling element 21.

It will further be clear to a skilled person that the catheter may be provided with various additional lumens and similar provisions which further improve the functionality of the catheter.

1. A catheter provided with a shaft having a proximal and distal end, and provided with a stenosis-removing part near the distal end of the shaft, wherein the stenosis-removing part is provided with hairs which can be brought into a first position and a second position, wherein, in a first position, the hairs substantially extend inside the circumferential contours of the shaft, wherein, in a second position, the hairs extend outside the shaft, so that the radial ends of the hairs are at a greater radial distance from the center line of the shaft than in the first position, wherein means are provided for making the hairs vibrate at a high frequency.

2. A catheter according to claim 1, wherein the means for making the hairs vibrate at a high frequency are designed for subjecting the hairs to a vibration frequency which is in the range of 10-100,000 Hz, more in particular in the range of 1000-50,000 Hz.

3. A catheter according to claim 2, wherein the hairs are connected with a holder, which holder is slidably connected with the shaft.

4. A catheter according to claim 3, wherein the holder comprises a bush-shaped body.

5. A catheter according to claim 4, wherein the catheter is provided with an insertion sleeve for keeping the hairs in the first position during the feeding of the stenosis-removing part towards the stenosis.

6. A catheter according to claim 5, wherein, proximal and optionally also distal of the stenosis-removing part, a filter is provided.

7. A catheter according to claim 6, wherein the means for making the hairs vibrate at a high frequency comprise a frequency generator and vibration transmission means designed for transmitting vibrations generated by a frequency generator to the hairs.

8. A catheter according to claim 7, wherein the vibration transmission means comprise a guide wire which is, by a distal end, couplable with the holder and which is, by a-proximal end, couplable with the frequency generators.

9. A catheter according to claim 8, wherein the holder is provided with at least one coupling wall or coupling pin inside the holder, which coupling wall or pin is provided with a central passage through which the guide wire can be fed, wherein the guide wire is, at the distal end, provided with a coupling element which is couplable with the coupling wall or pin of the holder.

10. A catheter according to claim 7, wherein the vibration transmission means comprise an actuator included in the shaft and arranged near the holder, wherein the actuator is connected with the frequency generator via communication means.

11. A catheter according to claim 10, wherein the communication means are electrical conductors extending in the shaft of the catheter.

12. A catheter according to claim 10, wherein the communication means comprise wireless communication means.

13. A catheter according to claim 10, wherein the actuator comprises a 25 coil and a coil core which is operatively connected to the holder.

14. A catheter according to claim 13, wherein the shaft is provided with a first lumen for feeding a guide wire through it.

15. A catheter according to claim 14, wherein the shaft is provided with a second lumen of which a distal opening is near the stenosis-removing part.

16. A catheter according to claim 15, wherein the shaft is provided with a third lumen of which a distal opening is near the stenosis-removing parts.

17. A catheter according to claim 16, wherein the distal openings of the second and the third lumen are distal of the proximal filter and proximal of the distal filter, if any, such that washing fluid in the area bounded by the filters can be supplied via the third lumen and can be discharged via the second lumen.

18. A catheter according to claim 17, wherein the catheter is provided with measuring means for measuring the vibration of the hairs, wherein evaluation means are provided for evaluating the measurements, wherein the evaluation means are connected to the frequency generator and wherein the evaluation results determine the frequency generated by the frequency generator.

18a. A catheter according to claim 17, wherein the catheter is provided with measuring means for measuring the vibration of the hairs, wherein evaluation means are provided for evaluating the measurements, wherein the evaluation means are connected to the frequency generator and wherein the evaluation results determine the amplitude generated by the frequency generator.

19. A catheter according to claim 18, wherein the measuring means are designed for measuring the vibration frequency and the vibration amplitude of the holder.

20. An assembly of a catheter according to claim 19, a guide wire, a frequency generator and, optionally, evaluation means.

21. A method for treating a stenosis in a blood vessel, wherein, a catheter according to claim 19 with insertion sleeve is slid into the blood vessel until the stenosis-removing part is near the stenosis, wherein, then, the insertion sleeve of the stenosis-removing part is removed, so that the hairs proceed from the first position to the second position, wherein, then, the means for making the hairs vibrate at a high frequency are switched on.

22. A method according to claim 21, wherein, during the treatment, the stenosis-removing part is moved from a distal part of the stenosis to a proximal part of the stenosis.

23. A method according to claim 22, wherein, after treating the whole stenosis, the catheter with the guide wire is removed from the blood vessel.

24. A method according to claim 23, wherein, prior to sliding the catheter into the blood vessel, first, a guide wire is slid into the blood vessel until the distal end of the guide wire has been slid beyond the stenosis, wherein, then, the catheter is slid over the guide wire.

25. A method according to claim 24, wherein, during the treatment, also, the vibration of the hairs is measured, wherein the measurements are evaluated and wherein, depending on the evaluation results, the frequency of the vibration is adjusted.

25a. A method according to claim 24, wherein, during the treatment, also, the vibration of the hairs is measured, wherein the measurements are evaluated and wherein, depending on the evaluation results, the amplitude of the vibration is adjusted.

26. A method according to claim 25, wherein, during the treatment, also, the result achieved during the treatment is determined, for instance by means of X-ray images or ultrasound, and wherein, depending on this determination, the treatment is continued, or terminated, or changed by changing frequency of the vibration or by replacing the catheter with a catheter having a different diameter.

26a. A method according to claim 25, wherein, during the treatment, also, the result achieved during the treatment is determined, for instance by means of X-ray images or ultrasound, and wherein, depending on this determination, the treatment is continued, or terminated, or changed by changing amplitude of the vibration or by replacing the catheter with a catheter having a different diameter.

27. A method according to claim 26, wherein the catheter is provided with at least one filter provided proximal of the stenosis-removing part, wherein, prior to removing the catheter, the area distal of the filter is exhausted via a second lumen of the catheter, of which second lumen, a distal opening is distal of the proximal filter.

28. A method according to claim 27, wherein, in the shaft of the catheter, a third lumen is provided, wherein a distal opening of the third lumen is distal of the proximal filter, wherein, during the exhaustion of the area distal of the proximal filter, washing fluid is supplied to this area via the third lumen.
